(19) United States
(12) Patent Application Publication
(10) Pub. No.: US 2011/0245190 A1
(43) Pub. Date: Oct. 6, 2011

(54) METHOD FOR IMPROVING POST SURGERY VISUAL ACUITY OUTCOME WITH AZITHROMYCIN

(76) Inventors: Romulus K. Brazzell, Morrisville, NC (US); Reza M. Haque, Randolph, NJ (US); Kurt E. Brubaker, Eagle Point, OR (US); Robert J. Dempsey, Middleton, MA (US); Joseph B. Boyd, Scottsdale, AZ (US)

(21) Appl. No.: 13/099,303

(22) Filed: May 2, 2011

Related U.S. Application Data
(63) Continuation-in-part of application No. 12/691,575, filed on Jan. 21, 2010.
(60) Provisional application No. 61/147,013, filed on Jan. 23, 2009.

(51) Int. Cl. A61K 31/7042; A61P 31/04; A61P 27/02

(52) U.S. Cl. 514/29

(57) ABSTRACT

The present invention relates to a method for treating dry eye disease, comprising the steps of identifying a subject suffering from dry eye disease, and administering to the subject an effective amount of azithromycin. The present invention also relates to a method for reducing contact lens intolerance in a subject, comprising the steps of: identifying a subject suffering from contact lens intolerance due to dry eye disease; and administering to the eyes of the subject an aqueous ophthalmic solution comprising azithromycin. The present invention also relates to a method for improving post surgery visual acuity outcome in a patient, comprising the steps of: identifying a patient in need of an ophthalmic surgical procedure, administering to the patient an effective amount of azithromycin prior to the ophthalmic surgical procedure, and performing the ophthalmic surgical procedures on the patient.

This application is a continuation-in-part of U.S. application Ser. No. 12/691,575, filed Jan. 21, 2010, which claims priority to U.S. Provisional Application No. 61/147,013, filed Jan. 23, 2009; the contents of the above-identified applications are incorporated herein by reference in their entirety.

### TECHNICAL FIELD

This invention relates to a method of treating dry eye disease in a subject. The method is useful in relieving dry eye signs and symptoms. The method involves administering to the subject in need thereof azithromycin. The present invention is illustrated by administering to the subject an azithromycin ophthalmic solution, such as AZASITE®.

### BACKGROUND OF THE INVENTION

Dry eye disease is the general term for indications produced by abnormalities of the precorneal tear film characterized by a decrease in tear production or an increase in tear film evaporation, together with the ocular surface disease and symptoms that result. Approximately 38 million Americans are affected with some type of dry eye disorder. Among the indications that are referred to by the general term “dry eye disease” are: keratoconjunctivitis sicca (KCS), age-related dry eye, Stevens-Johnson syndrome, Sjogren's syndrome, ocular cicatrical pemphigoid, corneal injury, infection, Riley-Day syndrome, congenital alacrima, nutritional disorders or deficiencies (including vitamins), pharmacologic side effects, contact lens intolerance, eye stress and glandular and tissue destruction, environmental exposure to smog, smoke, excessively dry air, airborne particulates, autoimmune and other immunodeficient disorders, and comatose patients rendered unable to blink.

Dry eye disease, although seen pathologically during ophthalmic exams as superficial punctate keratopathy (SPK) of the ocular surface epithelium, is largely a symptomatic disease. Chronic dryness leads to pain and irritation that is often debilitating to the subject, preventing the performance of normal daily activities such as reading, driving, etc. Dry eye is most common in postmenopausal women; however, hormone replacement therapy has not been proven to help dry eye signs and symptoms.

Contact lens wear is commonly associated with symptoms of ocular irritation including symptoms such as dryness, discomfort, soreness, tiredness, and irritation, among other symptoms. Reports have shown that between 25-50% of contact lens wearers have these symptoms, albeit of differing frequencies and severities. Further, these symptoms have been shown to increase in their intensity or severity over the course of a day's wear of lenses, with an average increase of approximately 25% as demonstrated in at least one study. (Dougherty et al. Br. J. Ophthalmol. 1984;68:524-8) Currently, the pharmaceutical treatment of dry eye disease is mostly limited to administration of artificial tears (saline solution) or anti-inflammatory agents (cyclosporine, steroids). Secretagogues (diquafosol, 15-HETE, rebamipide) to increase the production of tears are currently under development. In addition, artificial tears often have contraindications and incompatibility with soft contact lenses (M. Lemp, Cornea 9(1), S48-550 (1990)). The use of phosphodiesterase inhibitors, such as 3-isobutyl-1-methylxanthine (IBMX) to stimulate tear secretion is disclosed in U.S. Pat. No. 4,753,945. The effectiveness of these phosphodiesterase inhibitors has been investigated (J. Gilbard, et al., Arch. Ophthal, 112, 1614-16 (1994) and 109, 672-76 (1991); idem, Inv. Ophthal. Vis. Sci. 31, 1381-88 (1990)). Stimulation of tear secretion by topical application of melanocyte stimulating hormones is described in U.S. Pat. No. 4,868,154. Although these interventions can reduce inflammation and/or reduce SPK associated with dry eye, they have not been proven to significantly reduce the symptoms of dry eye.

Dry eye disease is different from blepharitis; the two diseases have different patient populations. Dry eye symptoms are dryness, burning, photophobia, foreign body sensation and grittiness in the eyes. Dry eye disease is characterized by an insufficient or defective tear film. The primary end points for studying dry eye diseases are corneal and conjunctival staining, measuring tear volume (Schirmer tests), Tear break-up time, dryness, burning, photophobia, foreign body sensation and grittiness.

Blepharitis is a chronic disorder producing inflammation of the anterior and posterior eyelid margin, with involvement of skin and its related structures (hairs and sebaceous glands), the mucocutaneous junction, and the meibomian glands (American Academy of Ophthalmology Preferred Practice Pattern 2003; Thygeson 1946; Foulks 2003). Blepharitis has historically been treated on a chronic basis (Dougherty 1984) through either mechanical therapy (consisting of improved eyelid hygiene and eyelid compression) alone or in combination with topical or systemic antibiotics. Typical clinical signs of blepharitis include lid debris, redness of eyelid margin, eyelid swelling, plugging of the meibomian gland, and obstructed meibomian gland secretion. The primary endpoints for studying blepharitis include the amount of eyelid debris and hyperemia of the eyelid margin.

Azithromycin is a macrolide antibiotic. AZASITE® (azithromycin ophthalmic solution) is a 1% sterile aqueous topical ophthalmic solution of azithromycin formulated in DURASITE® (polycarbophil, edetate disodium, sodium chloride). AZASITE® is approved by the U.S. Food and Drug Administration (FDA) for treatment of bacterial conjunctivitis, caused by susceptible isolates of CDC coryneform group G, Haemophilus influenzae, Staphylococcus aureus, Streptococcus mitis group, and Streptococcus pneumoniae (AZASITE® Package Insert, 2008).

As a result of the ineffectiveness and inconvenience of current therapies of dry eyes, there remains a need for a method of treating dry eye disease, which is not only effective, but also free of significant side effects.

### SUMMARY OF THE INVENTION

The present invention is directed to a method of treating dry eye disease or reducing one or more dry eye signs and/or symptoms in a subject in need of such treatment. The method comprises the step of first identifying a subject suffering from dry eye disease or dry eye symptoms, then administering to the subject an effective amount of azithromycin.

Particularly, the present invention is suitable for treating dry eye diseases caused by one or more of keratoconjunctivitis sicca, age-related dry eye, Stevens-Johnson syndrome, Sjogren's syndrome; ocular cicatrical pemphigoid, corneal injury, infection, Riley-Day syndrome, congenital alacrima, nutritional disorders or deficiencies, pharmacologic side effects, contact lens intolerance, eye stress resulting in glandular and tissue destruction, autoimmune disorders, immuno-deficient disorders, comatose patients who are unable to blink, or environmental exposure to smog, smoke, excessively dry air, or airborne particulates. The present invention is also suitable to treat patients suffering from dry eye disease, but not suffering from blepharitis.

The present invention is also directed to a method for reducing contact lens intolerance in a subject. The method comprises the steps of: identifying a subject suffering from contact lens intolerance due to dry eye disease; and administering to the eyes of the subject an aqueous ophthalmic solution comprising: (a) an active pharmaceutical ingredient consisting essentially of azithromycin, and (b) a physiologically compatible ophthalmic vehicle. The present method increases comfortable contact lens wearing time per day in the subject.

The present invention is also directed to a method for improving post surgery visual acuity outcome in a patient, which comprises the steps of: identifying a patient in need of an ophthalmic surgical procedure, administering to the patient an effective amount of azithromycin prior to the ophthalmic surgical procedure, and performing the ophthalmic surgical procedures on the patient.

Azithromycin is preferably administered topically to the subject in an aqueous ophthalmic solution comprises 0.5-1.5% (w/v) azithromycin in a polymeric suspension.

### BRIEF DESCRIPTION OF THE DRAWINGS

FIG. 1 compares the comfortable contact lens wear time in subjects treated with 1% azithromycin vs. rewetting drops.

FIG. 2 compares subject-rated ocular dryness (AM) in subjects treated with 1% azithromycin vs. rewetting drops. Each subject recorded the data prior to instilling study medication in the morning.

FIG. 3 compares subject-rated ocular dryness (PM) in subjects treated with 1% azithromycin vs. rewetting drops. Each subject recorded the data prior to removing contact lenses in the afternoon.

FIG. 4 show the study schematic design of pre-surgery treatment and post-operative visits.

### DETAILED DESCRIPTION OF THE INVENTION

The present invention is directed to a method of treating dry eye disease or reducing dry eye signs and/or symptoms such as dryness, burning, photophobia, foreign body sensation and grittiness in a subject. The present invention is also directed to a method of improving the tear film in a subject suffering from dry eye symptoms. The method comprises the step of first identifying a subject suffering from dry eye disease or dry eye symptoms, then administering to the subject an effective amount of azithromycin. In one embodiment, azithromycin is administered in an aqueous ophthalmic solution comprising no additional active pharmaceutical ingredient.

The inventors have discovered that azithromycin reduces signs and symptoms of dry eye and improves the tear film in patients with dry eye disease such as contact lens-related dry eye disease. The method of the present invention is an improvement upon the current most commonly used treatment of dry eye disease: artificial tears (i.e., saline solution) and anti-inflammatory agents (cyclosporine). A normal tear film is composed of a mucin layer, an aqueous component and a lipid layer. The present method improves the quality of a patient's own tear film. The present method also provides topical analgesia of the symptomatic corneal irritation that occurs in dry eye.

Dry eye symptoms can be due to one or more of keratoconjunctivitis sicca (KCS), age-related dry eye, contact lens intolerance, Stevens-Johnson syndrome, Sjogren's syndrome, ocular cicatrical pemphigoid, corneal injury, infection, Riley-Day syndrome and congenital alacrima. Dry eye symptoms can also be caused by nutritional (such as vitamin) disorders or deficiencies, autoimmune disorders, immuno-deficient disorders, pharmacologic side effects, eye stress resulting in glandular and tissue destruction, and environmental exposure to smog, smoke, excessively dry air, or airborne particulates.

The present invention is suitable for treating any dry eye diseases. Particularly, the present invention is suitable for treating dry eye diseases caused by one or more of keratoconjunctivitis sicca, age-related dry eye, Stevens-Johnson syndrome, Sjogren's syndrome, ocular cicatrical pemphigoid, corneal injury, infection, Riley-Day syndrome, congenital alacrima, nutritional disorders or deficiencies, pharmacologic side effects, contact lens intolerance, eye stress and glandular and tissue destruction, autoimmune disorders, immuno-deficient disorders, comatose patients who are unable to blink, or environmental exposure to smog, smoke, excessively dry air, or airborne particulates. The present invention is also suitable to treat patients suffering from dry eye disease, but not suffering from blepharitis. Subjects suitable for treatment by the present method often have had a clinical diagnosis of dry eye disease by a trained eye care professional.

The present invention is also useful in treating contact lens-related dry eye disease. The present invention reduces dry eye symptoms associated with contact lens wear in a subject who develops contact lens intolerance due to dryness in the eyes. The present invention enhances the number of hours of comfortable contact lens wearing time in a subject.

The present method comprises the steps of: (i) identifying a subject suffering from dry eye disease; and (ii) administering to the eyes of the subject an aqueous ophthalmic solution comprising: (a) an active pharmaceutical ingredient consisting essentially of azithromycin, and (b) a physiologically compatible ophthalmic vehicle; wherein said dry eye disease is caused by one or more of keratoconjunctivitis sicca, age-related dry eye, Stevens-Johnson syndrome, Sjogren's syndrome, ocular cicatrical pemphigoid, corneal injury, infection, Riley-Day syndrome, congenital alacrima, nutritional disorders or deficiencies, pharmacologic side effects, contact lens intolerance, eye stress resulting in glandular and tissue destruction, autoimmune disorders, immuno-deficient disorders, comatose patients who are unable to blink, or environmental exposure to smog, smoke, excessively dry air, or airborne particulates.

In another embodiment, the present method comprises the steps of: (i) identifying a subject suffering from dry eye disease and not suffering from blepharitis; and (ii) administering to the subject an aqueous ophthalmic solution comprising: (a) an active ingredient consisting essentially of azithromycin, and (b) a physiologically compatible ophthalmic vehicle.

In another embodiment, the present method comprises the steps of: (i) identifying a subject suffering from dry eye disease; and (ii) administering to the eyes of the subject an aqueous ophthalmic solution comprising: (a) active pharmaceutical ingredients consisting essentially of azithromycin and a secretegogue, and (b) a physiologically compatible ophthalmic vehicle. A secretegogue is a compound or agent that induces stored material to be released from cells or tissue. Examples of secretegogues include diquafosol, 15-HETE, or rebamipide.

The present invention is also directed to method for reducing contact lens intolerance in a subject. The method comprises the steps of: (i) identifying a subject suffering from contact lens intolerance due to dry eye disease; and (ii) administering to the eyes of the subject an aqueous ophthalmic solution comprising: (a) an active pharmaceutical ingredient consisting essentially of azithromycin, and (b) a physiologically compatible ophthalmic vehicle. The present method increases comfortable contact lens wearing time per day in the treated subject.

The present invention is further directed to a method for improving post surgery visual acuity outcome in a patient. The method comprises the steps of: (a) identifying a patient in need of an ophthalmic surgical procedure, (b) administering to the patient an effective amount of azithromycin prior to the ophthalmic surgical procedure, and (c) performing the ophthalmic surgical procedures on the patient, whereby the visual acuity of the patient is improved after the ophthalmic surgical procedures in comparison with what would have been without said administration of azithromycin prior to the surgical procedure.

In one embodiment, the patient in need of the ophthalmic surgical procedure has dry eye disease or is at the risk of developing dry eye disease. In another embodiment, the patient in need of the ophthalmic surgical procedure has blepharitis or is at the risk of developing blepharitis. Types of surgeries beneficial for the azithromycin pre-treatment include cataract surgery, refractive surgery such as PRK and Lasik, glaucoma surgery, corneal transplantation (keratoplasty), chalazion (acute or chronic eye lid lump) surgery, and pterygium (growth in cornea) surgery.

The subject benefits from the pretreatment with azithromycin before surgery because the azithromycin pretreatment improves the health of the ocular surface and quality of the lipid layer of the tear film. Azithromycin pretreatment improves the tear film quality by improving the meibomian gland secretions (which provide the lipid layer to the tear) from the eyelid. Azithromycin pretreatment provides tear film stability, which reduce dry eye.

After the azithromycin pretreatment, a surgeon measures a healthier eye in the treated subject when determining the procedures to perform during surgery. For example, when the surgeon measures a pre-treated eye pre-surgery to determine which replacement lens to use during cataract surgery or how much to correct the vision during Lasik surgery, the surgeon is measuring an eye with a healthier eye surface and better quality tear film and thus the eye surface is more like how the eye surface will be post surgery. This results in a better visual acuity outcome post surgery. With the azithromycin pretreatment prior to surgery, the subject improves his quality of life post surgery by reducing the dry eye or blepharitis symptoms, improving visual acuity, and reducing corneal epithelial defect.

The topical pretreatment of a patient with azithromycin before ophthalmic surgery is generally administered for a period of time commenced at least 7, 10, 14, 28, or 35 days prior to ocular surgery. The pretreatment generally continues for at least 7, 14, 21, or 28 days (preferably 14-28 days). The pretreatment typically stops 1-7 days, 1-4 days, 1-3 day, 1-2 days, or 1 day before surgery. For example, the patient is first pre-treated with azithromycin for 2-5 weeks (preferably 14-28 days), then the pre-treatment is stopped 1-2 days before the surgery is performed.

Many patients develop dry eye symptoms following ophthalmic surgery. The present invention is further directed to a method for treating dry eye diseases in a subject following an ophthalmic surgical procedure. The method comprises the steps of: identifying a subject suffering from dry eye disease following an ophthalmic surgical procedure, and administering to the subject azithromycin.

After treatment by the present methods, one or more dry eye signs and/or symptoms are reduced or alleviated in the subject. Dry eye symptoms include dryness, burning, ocular itching, photophobia, foreign body sensation, and grittiness. Dry eye signs are assessed by measurements such as: corneal and/or conjunctival staining (using fluorescein, lissamine green or rose Bengal stain), Shirmer's strip testing, Zone-Quick threads, tear film osmolarity, tear break-up-time and tear meniscus height.

The present invention is useful as a wash or irrigation solution to eyes of those who are unable to blink, for example, patients who cannot blink due to muscle or nerve damage, neuromuscular blockade or loss of the eyelids, comatose patients, or conscious individuals during surgery.

The “effective amount” of azithromycin administered to a subject is an amount effective to reduce the clinical signs and/or symptoms of dry eye disease. The present invention is not limited to the use of free base of azithromycin, it also includes the use of pharmaceutically acceptable salts of azithromycin. Pharmaceutically acceptable salts are salts that retain the desired biological activity of azithromycin and do not impart undesired toxicological effects.

The present invention is concerned primarily with the treatment of human subjects, but can also be employed for the treatment of other mammalian subjects, such as dogs and cats, for veterinary purposes.

Azithromycin can be administered to the eyes of a patient by any suitable means, including topical administration and systemic administration.

For topical administration, azithromycin is administered to the ocular surface of a subject, in an amount effective to reduce dry eye symptoms and to improve the tear film. Preferably, azithromycin is administered as a liquid or gel suspension in the form of drops, spray or gel. Alternatively, azithromycin can be applied to the eye via liposomes. Further, azithromycin can be infused into the tear film via a pump-catheter system. Azithromycin can also be contained within a continuous or selective-release device, for example, membranes such as, but not limited to, those employed in the Ocusert™ System (Alza Corp., Palo Alto, Calif.). Azithromycin can also be contained within, carried by, or attached to contact lenses or other compatible controlled release materials, which are placed on the eye. Azithromycin can also be contained within a swab or sponge which can be applied to the ocular surface. Azithromycin can also be contained within a liquid spray which can be applied to the ocular surface. Another embodiment of the present invention involves an injection of azithromycin directly into the lacrimal tissues or onto the eye surface.

The topical solution containing azithromycin can contain a physiologically compatible vehicle, as those skilled in the ophthalmic art can select using conventional criteria. The ophthalmic vehicles include, but are not limited to, saline solution, artificial tears, water polyethers such as polyethylene glycol, polyvinyls such as polyvinyl alcohol and povidone, cellulose derivatives such as methylcellulose and hydroxypropyl methylcellulose, polycarbophil, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

The topical formulation optionally includes a preservative, such as benzalkonium chloride and other inactive ingredients such as EDTA. The pH of the formulation is adjusted by adding any physiologically and ophthamologically acceptable pH adjusting acids, bases or buffers to within the range of about 5 to 7.5; preferably 6 to 7. Examples of acids include acetic, boric, citric, lactic, phosphoric, hydrochloric, and the like, and examples of bases include sodium hydroxide, sodium phosphate, sodium borate, sodium citrate, sodium acetate, sodium lactate, tromethamine, THAM (trishydroxymethylamino-methane), and the like. Salts and buffers include citrate/dextrose, sodium bicarbonate, ammonium chloride and mixtures of the aforementioned acids and bases.

The osmotic pressure of the topical formulation of azithromycin is generally from about 200 to about 400 milliosmolar (mOsM), more preferably from 260 to 340 mOsM. The osmotic pressure can be adjusted by using appropriate amounts of physiologically and ophthamologically acceptable ionic or non-ionic agents. Sodium chloride is a preferred ionic agent, and the amount of sodium chloride ranges from about 0.01% to about 1% (w/v), and preferably from about 0.05% to about 0.45% (w/v). Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can be used in addition to or instead of sodium chloride to achieve osmolality within the above-stated range. Further, non-ionic agents such as mannitol, dextrose, sorbitol, glucose and the like can also be used to adjust the osmolality.

The concentration of azithromycin included in the topical formulation is an amount sufficient to reduce dry eye symptoms and/or improve the tear film. This formulation is preferably an aqueous solution of azithromycin and is in the range of 0.005-3%, preferably 0.01% to 2%, preferably 0.1-2%, more preferably 0.5-1.5%, and most preferably about 1.0% (w/v). “About” as used herein, refers to ±15% of the recited value. The formulation optionally includes a preservative, such as benzalkonium chloride (0.003% w/v) and inactive ingredients: edetate sodium, purified water, sodium chloride, sodium phosphate monobasic, sodium hydroxide, and/or hydrochloric acid to adjust the pH to about 6-8, preferably about 7.

A preferred ophthalmic formulations of azithromycin suitable for the present method are those disclosed in U.S. Pat. Nos. 6,239,113, 6,569,443 and 7,056,893; the formulations of which are incorporated herein by reference. For example, the formulation is an aqueous polymeric suspension comprising water, azithromycin, and 0.1 to 10% of a polymeric suspending agent. The polymeric suspending agent comprises a water-swellable water-insoluble crosslinked carboxy-vinyl polymer. For example, the polymeric suspending agent comprises least 90% acrylic acid monomers and 0.1% to 5% crosslinking agent. AZASITE® (azithromycin ophthalmic solution), which is a 1% sterile aqueous topical ophthalmic solution of azithromycin formulated in DURASITE® (polycarbophil, edetate disodium, sodium chloride), is the most preferred ophthalmic formulation. The preferred ophthalmic formulations are able to keep prolonged high azithromycin concentration on the ocular surface, thus facilitating its penetration into the eye tissues.

The daily topical dose to reduce dry eye symptoms and improve tear film composition can be divided among one or several unit dose administrations. The total daily dose for azithromycin, for example, can range from one drop (about 50 one to four times a day, depending upon the age and condition of the subject. A preferred regimen for azithromycin is one drop of 1.0% (w/v) solution, about 1 to 2 times a day.

In addition to the topical method of administration described above, there are various methods of administering azithromycin systemically. One such method involves an aerosol suspension of respirable particles comprised of azithromycin, which the subject inhales. Azithromycin is absorbed into the bloodstream via the lungs or via nasolacrimal ducts, and subsequently contact the lacrimal glands in a pharmaceutically effective amount. The respirable particles can be liquid or solid, with a particle size sufficiently small to pass through the mouth and larynx upon inhalation; in general, particles ranging from about 1 to 10 microns, but more preferably 1-5 microns, in size are considered respirable.

Liquid pharmaceutical compositions of azithromycin for producing a nasal spray or nasal or eye drops can be prepared by combining azithromycin with a suitable vehicle, such as sterile pyrogen free water or sterile saline by techniques known to those skilled in the art.

Other method of systemic administration of the active compound involves oral administration, in which pharmaceutical compositions containing azithromycin are in the form of tablets, lozenges, aqueous or oily suspensions, dispersible powders or granules, emulsion, hard or soft capsules, or syrups or elixirs. Compositions intended for oral use can be prepared according to any method known to the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain azithromycin in admixture with nontoxic pharmaceutically acceptable excipients, which are suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

A preferred systemic administration is oral administration. In a tablet, a preferred dose contains 1-2000 mg, preferably 50-1000 mg and most preferably 250-500 mg of azithromycin, and is administered once or twice a day. Alternately, an oral syrup or dry syrup such as 1-2 teaspoons of a 1% (w/v) azithromycin suspension can be administered to a subject once or twice a day.

Additional method of systemic administration of azithromycin to the eyes of the subject involves a suppository form of the active compound, such that a therapeutically effective amount of the compound reaches the eyes via systemic absorption and circulation.

Further method of administration of azithromycin involves direct intra-operative instillation of a gel, cream, powder, foam, crystal, liposomes, spray or liquid suspension form of said compound, such that a therapeutically effective amount of the compound reaches the eyes via systemic absorption and circulation.

The invention is illustrated further by the following example, which is not to be construed as limiting the invention to the specific procedures described in it.

### EXAMPLES

### Example 1

### Use of Azithromycin for Reducing Symptoms in Patients with Dry Eye Disease

### Objectives

The objective of this study is to compare the efficacy of study drug, AZASITE® (azithromycin ophthalmic solution) 1%, versus the vehicle (DuraSite®) over a four week treatment period on the symptoms of subjects with dry eye disease.

### Subjects

Subjects are 18 years of age or older, and have a clinical diagnosis of mild to moderate dry eye disease. A total of 100 subjects are enrolled in the study.

### Methods

This is a double-masked study. At Visit 1 (Day 1), all subjects are randomized in 1:1 ratio to receive either (a) AZASITE® or (b) the vehicle DURASITE®), for 30 days. Study drug is administered as one drop in each eye BID for the first 2 days and then QD for the remainder of the study. Study drugs are self-administered by the subjects. The subjects are prohibited in using any ocular or other medications that could confound the results of the assessments during study participation, such as antihistamines, steroids, antibiotics or preserved artificial tears.

Patients return for Visit 2 (Day 14), and Visit 3 (Day 30) and are asked to rate their symptoms including: ocular itching, ocular burning/pain, and foreign body sensation.

### Efficacy Assessment

Efficacy endpoints (dry eye signs are symptoms) are assessed at Visit 3:
Change from baseline of Tear Break-Up Time (TBUT)
Clearing of Inferior Fluorescein Corneal Staining
Clearing of Total Fluorescein Corneal Staining
Change from baseline of Tear Volume
Change from baseline of investigator-rated severity of meibomian gland plugging
Change from baseline of subject-rated scores of dry eye symptoms of eyelid itching, foreign body sensation/sandiness, grittiness, and ocular burring and pain.

Patients rate the severity of their dry eye symptoms at Visits 1, 2, and 3 according to the following three classifications.

### Eyelid Itching

Do your eyelids feel itchy?

(0) None: My eyelids do not feel itchy.

(1) Mild: Once in a while, my eyelids feel slightly itchy, but I do not have a desire to rub them.

(2) Moderate: Occasionally, my eyelids feel itchy, and I need to rub them.

(3) Severe: It is difficult to relieve the sensation of itchiness even when I rub my eyelids.

(4) Very severe: I have unbearable eyelid itching with an irresistible urge to rub my eyelids.

### Foreign Body Sensation/Sandiness, Grittiness

Do you feel like there's something sandy or gritty in your eye?

(0) None: My eyes do not feel sandy or gritty.

(1) Mild: I am aware of the surface of my eyes once in a while.

(2) Moderate: My eyes feel like there is something small in them occasionally.

(3) Severe: My eyes feel like there is something large or gritty in them.

(4) Very severe: I am unable to open my eyes due to feeling of a foreign body in my eyes.

### Ocular Burning or Pain

Are your eyes burning or painful?

(0) None: My eyes do not burn or ache.

(1) Mild: I am aware of the surface of my eyes; they mildly burn or ache.

(2) Moderate: I feel my eyes are burning, but still tolerable

(3) Severe: My eyes feel throbbing or fiery due to burning/pain.

(4) Very severe: I am unable to open my eyes due to burning/pain

### Results

The mean scores for individual symptoms for each group (AZASITE® and DURASITE®), are compared for Visits 2-3 to baseline (Visit 1). A statistically significant difference (p<0.05) is observed in favor of the AZASITE® treatment group for at least one of the Visits.

### Conclusions

The above results indicate that AZASITE® improves the symptoms of dry eye disease significantly greater than the vehicle, DURASITE®.

### Example 2

### Use of Azithromycin for Treating Patients With Contact Lens Related Dry Eye Disease

### Objectives

The objective of this study is to compare the efficacy of study drug, AZASITE® (azithromycin ophthalmic solution) 1%, versus rewetting drops (Visine for Contacts™) over a four week treatment period in patients with contact lens-related dry eye disease (CLDE).

### Subjects

Subjects are 18 years of age or older. Subjects have a positive diagnosis of CLDE based on their responses to the Contact Lens Dry Eye Questionnaire (CLDEQ, Nichols et al, Cornea 21:469-75 (2002)) and a score of ≧2, (i.e., moderate severity on a 0-4 scale, where 0=no ocular dryness and 4=very severe ocular dryness) on a self-reported assessment of ocular dryness at the screening Visit. A total of 50 subjects are enrolled in the study.

### Methods

### Visit 1 (Day 1)

Subjects were asked to rate their level of ocular dryness on a 0-4 scale. Tear osmolarity was then assessed for each eye using the TearLab Osmolarity System®. Subjects were then instructed to remove their contact lenses and slit-lamp biomicroscopy and external eye exams were performed. Fluorescein corneal staining was then performed on each eye (5 μl of 2% fluorescein applied to the ocular surface) and graded according to National Eye Institute (NEI) criteria.

Eligible subjects were then randomized to receive either azithromycin 1% ophthalmic solution (AzaSite®), or rewetting drops (Visine for Contacts™) in a 1:1 ratio and they were not masked as to the treatment assignment. Subjects randomized to azithromcyin 1% were instructed to administer one drop to each eye BID for the first two days and then one drop QD for days 3-29. Subjects randomized to rewetting drops were instructed to administer one to two drops QID throughout the 29 day treatment period. The first dose of study medication was self-administered at the clinic once dosing instructions were provided. Subjects were instructed to apply a new pair of contact lenses to their eyes 10 minutes after the instillation of study medication. Subjects were queried for adverse events (AEs) and scheduled to return for Visit 2 in approximately two weeks.

Subjects were instructed to record the AM ocular dryness assessment prior to instilling study medication (and lens application), and record PM dryness levels prior to lens removal each day. Further, subjects were required to record the time that contacts were inserted, the time lenses became uncomfortable and the time lenses were removed.

### Visit 2 (Day 14±2)

Concomitant medication usage was recorded, an AE query performed, study medication and diary cards were collected. Subjects underwent habitual HCVA and LCVA assessment, completed the CLDEQ, and had tear osmolarity readings measured prior to removing their contact lenses. Biomicroscopy, external eye exam, and fluorescein corneal staining were subsequently performed. The subjects then reapplied their lenses and additional study medication and diary cards were provided.

### Visit 3 (Day 29±1)

All assessments and procedures were performed the same manner as at Visit 2.

### Statistical Analyses

The primary efficacy endpoint for this study was the change from baseline in the duration of subject-reported comfortable contact lens daily wear time (hours/day) at the Week 4 Endpoint. The Week 4 Endpoint was defined as the average of the change from baseline values for comfortable contact lens daily wear time from days 23 to 29.

Secondary efficacy endpoints from the data captured from the diaries included: change from baseline in comfortable contact lens wear time at Week 1 (days 2-8), Week 2 (days 9-15), Week 3 (days 16-22), change from baseline in total contact lens wear time (Week 1-4 time intervals as defined for comfortable contact lens wear), and change from baseline in subject-reported levels of ocular dryness both at the morning (AM) assessment and evening (PM) assessment.

### Results

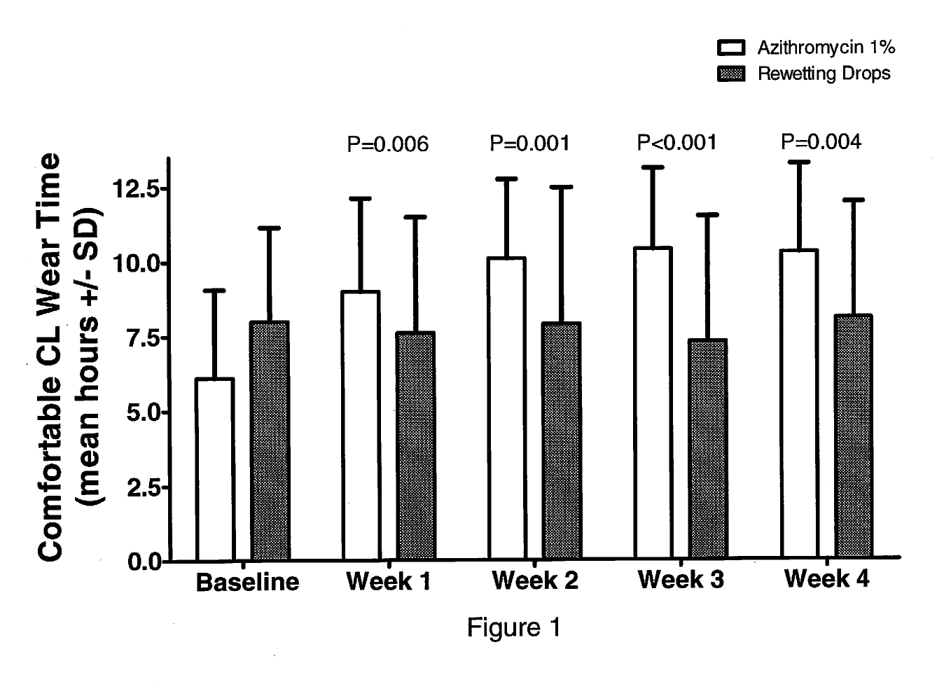

The mean daily duration of comfortable contact lens wear time was longer for subjects treated with azithromycin than rewetting drops at each of the weekly endpoints (Weeks 1-4) after initiating treatment (FIG. 1). Significant differences were observed between treatment groups for the change from baseline in the duration of comfortable contact lens time not only at the primary endpoint, Week 4 Endpoint (p=0.004), but also at the Weeks 1-3 (p=0.006, p=0.001, p<0.001, respectively) endpoints.

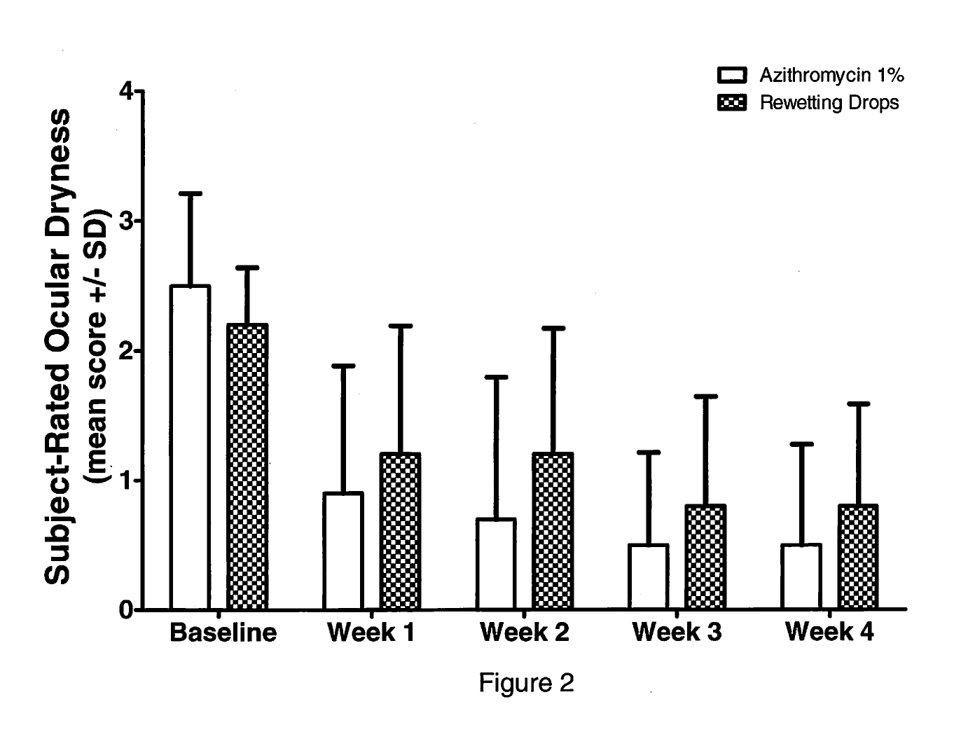

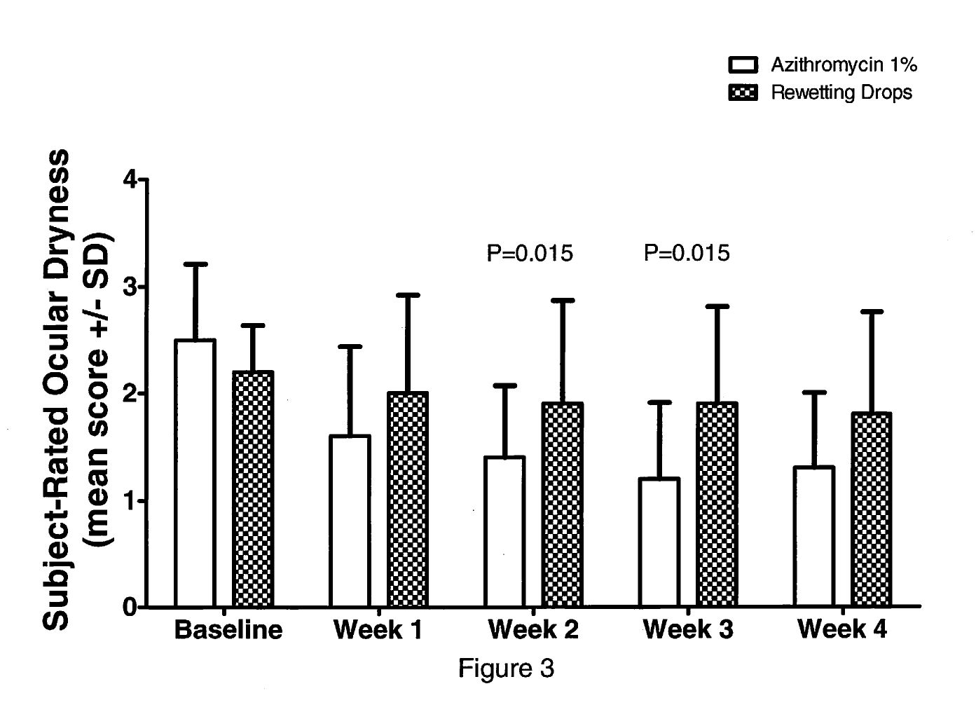

Subject-rated levels of ocular dryness were reduced in both groups following initiation of treatment. Mean ocular dryness scores of subjects treated with azithromcyin were lower than those treated with rewetting drops at both the AM (FIG. 2) and PM assessments (FIG. 3). Statistically significant differences in the change from baseline levels of ocular dryness between treatment groups were only observed at the PM assessment. At Weeks 2 and 3, a statistically significant reduction in ocular dryness (PM assessment) was observed for subjects treated with azithromycin as compared to the change from baseline for subjects treated with rewetting drops (p=0.015 for both endpoints).

### Example 3

### Post-Surgical Visual Outcome in Patients Treated With Azithromycin Prior to Surgery

### Objectives

The objective of this study is to compare the safety and efficacy of azithromycin ophthalmic solution, 1% to artificial tears in subjects with blepharitis over a four-week treatment period prior to cataract surgery by evaluating improvements in blepharitis, pre-operative lens selection, corneal topography and subsequent improvements in post-surgical outcomes related to visual acuity.

### Study Population

The study population consists of approximately 60 subjects with a diagnosis of both blepharitis and a cataract. Eligible subjects who elect to have cataract surgery for a premium multifocal intraocular lens (IOL) are recruited for this study. These subjects are enrolled at approximately three centers across the United States.

### Inclusion Criteria

Individuals of either gender or any race are eligible for study participation if they:

1. Have provided written informed consent prior to any study procedures.

2. Are 55 years of age or older.

3. If female, are non-pregnant, non-lactating and those of childbearing potential must be using an acceptable method of birth control [e.g., an Intrauterine Contraceptive Device with a failure rate of <1%, hormonal contraceptives, or a barrier method] for the duration of the study. If a female subject is currently abstinent, they must agree to use one of the acceptable methods of birth control before they become sexually active.

4. Are willing and able to follow instructions and can be present for the required study visits for the duration of the study.

5. Have juvenile or senile cataract.

6. Have a corneal astigmatism in surgery eye of ≦1.0 diopter (D) as measured on IOL master.

7. Have a diagnosis of blepharitis in eye scheduled for cataract surgery (the surgery eye) which meets both of the following criteria:
A clinical sign severity score of at least 1 (mild) on Meibomian Gland plugging.
A clinical sign severity score of at least 1 (mild) on Lid Debris

8. Elect to have cataract surgery for a premium IOL (multifocal).

### Exclusion Criteria

Individuals are not eligible for study participation if they:

1. Have presence of inflammation and/or active structural change in any ocular structure (other than the eyelid margin and lens).

2. Have lid structural abnormalities such as entropion or ectropion.

3. Have suspected ocular fungal or viral infection.

4. Have had penetrating intraocular surgery in the 90 days prior to Visit 1 or require penetrating intraocular surgery during the study.

5. Have had ocular surface surgery (e.g. Laser-assisted in situ keratomileusis (LASIK), refractive, pterygium, etc.).

6. Have intraocular pressure measurements ≧22 mmHg in either eye at any time point.

7. Have used topical ocular or oral antibiotics within 30 days prior to Visit 1.

8. Have any documented previous use of isotretinoin (Accutane®) within 30 days prior to Visit 1 or plan to use this medication during the study.

9. Have used topical cyclosporine (Restasis®) within 30 days prior to Visit 1 or during the two treatment periods.

10. Have used systemic steroids, topical ocular steroids, or ocular non-steroidal anti-inflammatory drugs (NSAIDs) in eye scheduled for cataract surgery (the surgery eye) within 14 days prior to Visit 1 or during the two treatment periods.

11. Have used topical ocular or oral antihistamine and/or mast cell stabilizers within 14 days prior to Visit 1 or during the two treatment periods.

12. Have used any prostaglandin or prostaglandin analogue within 30 days prior to Visit 1 or during the two treatment periods.

13. Are currently using any topical ocular medications within 3 days prior to Visit 1 or during the two treatment periods.

14. Are unwilling to discontinue use of contact lenses during the study.

15. Have a known hypersensitivity to azithromycin, erythromycin, any other macrolide antibiotic, Refresh Tears® Lubricant Eye Drop or any of the other ingredients in either study medication and Acuvail™ or Pred Forte®.

16. Have a history of herpetic keratitis within the past year.

17. Have a history of amblyopia in the study eye.

18. Have a concomitant ocular pathology that, in the opinion of the investigator, may confound study assessments.

19. Have a serious systemic disease or uncontrolled medical condition that in the judgment of the investigator could confound study assessments or limit compliance.

20. Have been exposed to any investigational drug within 30 days prior to Visit 1.

21. Are an employee of the site that is directly involved in the management, administration, or support of this study or be an immediate family member of the same.

22. Are unwilling or unable to comply with the protocol.

### Study Design

This is a randomized, multi-center, open-label, parallel group, safety and efficacy study designed to compare the effects of four weeks of treatment with azithromycin ophthalmic solution, 1% versus artificial tears in subjects with blepharitis undergoing cataract surgery.

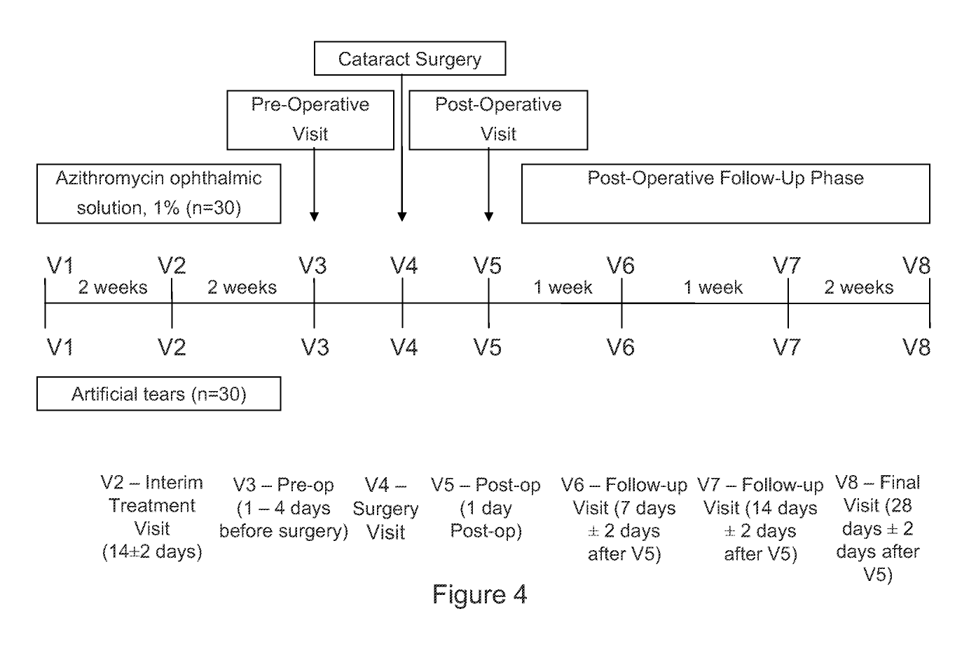

The study design includes eight clinic visits over eight weeks (see FIG. 4 for study schematic). Subject eligibility is established at the screening and randomization visit (Visit 1).

At Visit 1, eligible subjects who elect to have cataract surgery for a premium IOL (multifocal) are randomized 1:1 ratio to one of two therapy arms and initiate either treatment with azithromycin ophthalmic solution, 1% or artificial tears.

At Visit 1, all subjects randomized administer azithromycin ophthalmic solution, 1% or artificial tears into each eye BID for two days (beginning at Visit 1) then QD in the evening at approximately the same time of day for the remainder of the 28-day treatment period ending at Visit 3 (approximately 28 days on study drug treatment). Each subject is instructed to massage the upper and lower lids of both eyes for 1 minute after the administration of study drug.

Visit 2 is an interim treatment visit.

Visit 3 is a pre-operative (pre-op) visit. Subjects must not use study drug on the day of Visit 3. All subjects are treated with Zymaxid® (gatifloxacin ophthalmic solution) 0.5% QID starting immediately after Visit 3, and for 10 days post operative (post-op) and Acuvail™ (ketorolac tromethamine ophthalmic solution) 0.45% BID starting immediately after Visit 3 and for 4 weeks post-op. Additionally all subjects are treated with Pred Forte® (prednisolone acetate ophthalmic suspension, USP) 1% QID starting one day post-op and continuing for 4 weeks post-op.

Visit 4 is cataract surgical procedure visit.

Visit 5 occurs 1 day after Visit 4.

Visits 6-8 are follow-up visits at 7±2 days, 14±2 days and 28+2 days after Visit 5. Visit 8 is the final study visit. The total duration of the study is approximately 8-8½ weeks.

### Clinical Endpoints:

The efficacy endpoints for this study are to:
Evaluate a change in IOL selection from baseline on the surgery eye only
Evaluate improvements in Schirmer with anesthesia
Evaluate the mean and change from baseline in both eyes in the Tear Break-Up Time (TBUT)
Evaluate improvements in un-corrected visual acuity (UCVA)
Evaluate improvements in best corrected visual acuity (BCVA)
Evaluate the post-surgical (Visit 5) improvements in contrast sensitivity on the surgery eye using the Functional Acuity Contrast Test (F.A.C.T.®)
Evaluate changes from baseline in the Quality of Life Self-Assessment VF-7 (Visual Function-7) questionnaire responses
At baseline (Visit 1) and at each post-baseline study visit, evaluate and score each of the individual severity scores of the following clinical signs of blepharitis:
   Lid debris
   Erythema of eyelid margin
   Plugging of the meibomian gland
   Meibomian gland secretion
Determine the mean and change from baseline in both eyes for the above-identified investigator-rated scores of subjective clinical signs of blepharitis
At baseline (Visit 1) and at each post-baseline study visit, evaluate the individual subject-rated severity score of the symptoms of blepharitis:
   Foreign body sensation
   Ocular dryness
   Ocular burning/pain
   Swollen/heavy eyelids
   Eye itching
Determine the mean and change from baseline in both eyes for the above identified individual subject-rated scores of symptoms of blepharitis

### Safety Endpoints:

Safety is assessed on both eyes via:
Slit-lamp Biomicroscopy
Ophthalmoscopy and Intraocular Pressure (TOP)
AE monitoring

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A method for improving post surgery visual acuity outcome in a patient, comprising the steps of:
identifying a patient in need of an ophthalmic surgical procedure,
administering to the patient an effective amount of azithromycin prior to the ophthalmic surgical procedure, and
performing the ophthalmic surgical procedures on the patient, whereby the visual acuity of the patient is improved.

2. The method according to claim 1, wherein said patient in need of the ophthalmic surgical procedure has dry eye disease or is at the risk of developing dry eye disease.

3. The method according to claim 1, wherein said patient in need of the ophthalmic surgical procedure has blepharitis or is at the risk of developing blepharitis.

4. The method according to claim 1, wherein said ophthalmic surgical procedure is cataract surgery.

5. The method according to claim 1, wherein said ophthalmic surgical procedure is refractive surgery.

6. The method according to claim 1, wherein the azithromycin is in an aqueous solution comprising polycarbophil, edetate disodium, and sodium chloride.

7. The method according to claim 1, wherein the effective amount of azithromycin is 0.5-1.5% (w/v).

8. The method according to claim 5, wherein the effective amount of azithromycin is about 1% (w/v).

9. The method according to claim 1, wherein the patient is administered with azithromycin for 14-28 days, then the administration is stopped for 1-2 days prior to the ophthalmic surgical procedure,

10. A method for reducing contact lens intolerance in a subject, comprising the steps of:
(i) identifying a subject suffering from contact lens intolerance due to dry eye disease; and
(ii) administering to the eyes of the subject an aqueous ophthalmic solution comprising:
(a) an active pharmaceutical ingredient consisting essentially of azithromycin, and (b) a physiologically compatible ophthalmic vehicle.

11. The method according to claim 10, wherein said method increases comfortable contact lens wearing time per day in the subject.

12. The method according to claim 10, wherein said physiologically compatible ophthalmic vehicle comprises artificial tears.
